(19) United States
(12) Patent Application Publication
(10) Pub. No.: US 2025/0354917 A1
(43) Pub. Date: Nov. 20, 2025

(54) METHOD FOR THE QUANTIFICATION OF SODIUM IN PETROLEUM RESIDUES

(71) Applicant: PETRÓLEO BRASILEIRO S. A. - PETROBRAS, Rio de Janeiro (BR)

(72) Inventors: Rainerio Escalfoni Junior, Rio de Janeiro (BR); Bruno Rodrigues De Moura, Rio de Janeiro (BR); Ana Gabriela Apolonio De Souza, Rio de Janeiro (BR); Reginaldo Vicente, Rio de Janeiro (BR); Gaspar Barbosa Alexandre, Rio de Janeiro (BR)

(21) Appl. No.: 19/205,263

(22) Filed: May 12, 2025

(30) Foreign Application Priority Data
May 14, 2024 (BR) BR 1020240095170

(51) Int. Cl. G01N 21/31; G01N 31/00; G01N 33/28

(52) U.S. Cl. CPC G01N 21/31; G01N 31/005; G01N 33/2835

(57) ABSTRACT

The present invention relates to a direct method for the quantification of sodium in crude oil residues, such as atmospheric residues and vacuum residues by flame atomic absorption spectrometry (FAAS). The proposed method has application in the analysis of sodium in RAT, RV and more frequent oils for monitoring the efficiency of desalting in refineries and the effects of residual salt in other refining processes.

### FIELD OF THE INVENTION

The present invention is part of the Oil and Gas field, more precisely in the areas of coking, separation processes and refining technologies, and refers to a direct method for the quantification of sodium in crude oil residues, such as atmospheric residues and vacuum residues, by flame atomic absorption spectrometry (FAAS).

### BACKGROUND OF THE INVENTION

The quantification of salt content in crude oil is important to decide the extent of desalination required or the efficiency of this process. Most of the salt will be dissolved in coexisting free water and can be removed in desalters, but small amounts of salt may remain dissolved in the crude oil itself. Even in small concentrations, salts will accumulate and, during flash vaporization of oils, certain metal salts can be hydrolyzed to extremely corrosive hydrochloric acid, according to the following equations:

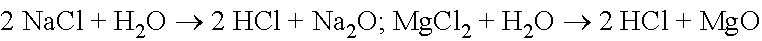

Excess chloride remaining in the desalted oil often results in high corrosion rates in subsequent refining units, having detrimental effects on the catalysts used in the refining process. The need for a faster method for quantifying sodium (Na) in heavy oils (such as vacuum residues, RV, or atmospheric residues, RAT) has become critical with the current processed cast of oils and the possible consequences of some residual salinity in the refining processes.

It is known that the quantification of sodium in samples of petroleum and heavy derivatives is normally done in methods where the organic fraction of the samples is oxidized using calcination and the determination of metals is performed by flame atomic absorption spectrometry (FAAS) or by inductively coupled plasma atomic absorption emission spectrometry (ICP-AES). This is a traditional and reliable analytical route, but it has a limitation in analysis time: the sample burning step can take from 8 to 40 hours depending on the type of sample, and which equipment is being used for the burning step.

There are approaches that involve the direct quantification of Na in petroleum involving dilution with solvents. ASTM D5863—Standard Test Methods for Determination of Nickel, Vanadium, Iron, and Sodium in Crude Oils and Residual Fuels by Flame Atomic Absorption Spectrometry (Test Method B) uses xylene, tetralin, or mixtures of paraffinic and aromatic solvents. However, the text of the standard itself warns that “this method uses oil-soluble metals for calibration to determine dissolved metals and is not intended to quantitatively determine or detect insoluble particles. Thus, this test method may underestimate the metal content, especially sodium, present as inorganic sodium salts”. The solvent mixture used in the proposed method (ethanol and xylene) in the tested proportion ensures satisfactory recovery of inorganic sodium in the tested samples (around 80%). The calibration of the atomic absorption spectrometer equipment is performed with standards prepared with the solvent mixture (xylene and ethanol) in the tested proportion from aqueous standards of sodium salts in aqueous medium.

### State of the Art

The document in the name of BARROS et al., entitled “DETERMINATION OF SODIUM AND POTASSIUM IN BIODIESEL BY FLAME ATOMIC EMISSION SPECTROMETRY, WITH DISSOLUTION IN ETHANOL AS A SINGLE SAMPLE PREPARATION STEP”, refers to the determination of sodium and potassium in biodiesel by flame atomic emission spectrometry (FAES), with dissolution in ethanol as a single sample preparation step. The document indicates that FAES can be a simple, fast and relatively inexpensive alternative analytical technique for the determination of Na and K in biodiesel. Ethanol is an excellent solvent for biodiesel and can replace toxic solvents such as xylene in sample preparation procedures by dissolution. The use of ethanol allows the use of aqueous stock solutions, thus avoiding the use of organometallic standards, which tend to be unstable.

The document in the name of Ariane Isis Barros, entitled “MÉTODOS ALTERNATIVOS PARA A DETERMINAçÃO DE SÓDIO, POTÁSSIO, CÁLCIO E MAGNÉSIO EM BIODIESEL” (ALTERNATIVE METHODS FOR DETERMINING SODIUM, POTASSIUM, CALCIUM AND MAGNESIUM IN BIODIESEL), refers to alternative methods for determining sodium, potassium, calcium and magnesium in biodiesel. Among the techniques used, flame atomic absorption spectrometry (FAAS) is mentioned.

According to the document, for the dissolution in ethanol method, the instrumental response and precision of the method are comparable to those obtained by the official Brazilian method. In addition, the proposed method is simple, involves few steps, uses low-cost solvent, low toxicity and aqueous standards that contribute to the stability of the analytical signal. Thus, dissolution in ethanol, as a sample preparation procedure, can be a useful alternative for determining Na, K, Ca and Mg in biodiesel by FAAS.

The document in the name of OLIVEIRA et al., refers to the determination of Na, K, Ca and Mg in biodiesel by FAAS using ethanol in sample preparation. According to the document, the proposed method is simple, fast and involves a single step in the sample preparation procedure, which contributes to the precision and accuracy of the results. In addition, the use of ethanol in sample preparation contributed to the reduction of the use of highly toxic solvents such as xylene, as well as to the reduction of analysis costs.

The document in the name of Conny Cerai Ferreira, entitled “DETERMINAÇÃO DIRETA E SIMULTÂNEA DE SÓDIO, POTÁSSIO E CÁLCIO EM BIODIESEL POR ESPECTROMETRIA DE EMISSÃO ATÔMICA EM CHAMA” (DIRECT AND SIMULTANEOUS DETERMINATION OF SODIUM, POTASSIUM AND CALCIUM IN BIODIESEL BY FLAME ATOMIC EMISSION SPECTROMETRY), refers to the direct and simultaneous determination of sodium, potassium and calcium in biodiesel by flame atomic emission spectrometry. Two possible calibration methods were proposed, the first being external calibration and the second external calibration using an internal standard to correct possible transport effects of the solutions. The calibration solutions were prepared by solubilizing organometallic standards in methyl oleate, which was used as a simulacrum of the matrix.

Thus, the mentioned documents from the state of the art describe methodologies applicable to biodiesel, a fuel defined as a mixture of esters resulting from the chemical process (transesterification) of oils and/or fats. It is a “final product”, being a type of fuel regulated by the National Petroleum Agency with the characteristics of an ester content of not less than 96.5% by mass, and a cold clogging point between 0° C. and 19° C., that is, they flow normally at temperatures of 20° C. (ANP RESOLUTION #920, OF Apr. 4, 2023). The present invention is directed to petroleum and distillation residues. These are complex mixtures of thousands of chemical compounds such as hydrocarbons (paraffins, aromatics, etc.) and others containing heteroatoms (asphaltenes, resins, etc.) and, in the case of RAT and RV, they are samples that do not flow at room temperature (they start to flow at 80° C. or more).

The current approach for analyzing metals in petroleum, RAT or RV (through burning and calcining the samples) is described in national and international standards and procedures. However, the quantification of sodium (Na) is not common, with the elements Ni, V, Fe, Al and Si normally being quantified. Therefore, a person skilled in the art would find and use sample preparation approaches involving burning and calcining the samples or acid digestion of them. The approach using direct sodium quantification for petroleum (and not for RAT or RV) is described in ASTM D5863—22 “Standard Test Methods for Determination of Nickel, Vanadium, Iron, and Sodium in Crude Oils and Residual Fuels by Flame Atomic Absorption Spectrometry” procedure B however there is the remark: “1.3 Procedure B, Sections 15-20-Flame AAS is used to analyze a sample diluted with an organic solvent for the determination of Ni, V, and Na. This test method uses oil-soluble metals for calibration to determine dissolved metals and does not purport to quantitatively determine nor detect insoluble particulates. The possible solvents used are listed below: “16.1 Dilution Solvent-Mixed xylenes, o-xylene, tetralin and mixed paraffin-aromatic solvents are satisfactory. (Warning-Combustible. Vapor harmful.) Solvent purity can affect analytical accuracy when the sample contains low concentrations (typically, a few mg/kg) of the analytes”. Again, there is no tendency to use ethanol or solvent mixtures applied to the types of samples included in the present application, nor there is a need to increase the solvency power of the samples and/or chemical compounds to be analyzed.

The present invention is based on a methodology that allows the characterization of sodium quickly and with satisfactory recovery. The use of a solvent mixture that is not used in any standard or bibliographic reference is proposed. The mixture used (xylene-ethanol) has the following objectives:
to completely solubilize the sample and the compounds of the element to be determined (in this case sodium): xylene completely solubilizes compounds present in petroleum, RAT and RV, while ethanol enables the dissolution of inorganic sodium species.
enable the use of inorganic standards of the element to be analyzed: thus, the calibration of the flame atomic absorption spectrometer (FAAS) equipment is performed with an inorganic compound of the element to be analyzed (in this case sodium) similar to that present in the samples (sodium chloride).
provide stability to this mixture: enabling the analysis to be performed without phase separation, loss of analytical signal and with complete burning in the FAAS equipment.

Nevertheless, the present invention involves: In the present case, the application involves (i) the use of the solvent mixture in the appropriate proportion: sufficient xylene to solubilize the sample and not in excess to impair the solubilization of sodium; sufficient ethanol to solubilize sodium without destabilizing the solubilization of the sample; (ii) the type(s) of sample analyzed and the quantification of sodium (unusual): the need for sodium quantification, especially in RAT and RV, is not yet a concern for industry and academia, and is related to the characteristics of the refining park and the range of processed oils.

The use of solvents eliminates the need for burning and calcining the samples, reducing the total time required to perform the test. The solvent mixture used ensures recovery of the sodium present in the sample. The calibration of the atomic absorption spectrometer equipment is performed with standards prepared with the solvent mixture (xylene and ethanol) in the tested proportion from aqueous standards of sodium salts in aqueous medium.

### SUMMARY OF THE INVENTION

The present invention aims to propose a direct method for the quantification of sodium in crude oil residues, such as atmospheric residues and vacuum residues, by flame atomic absorption spectrometry (FAAS).

### BRIEF DESCRIPTION OF THE INVENTION

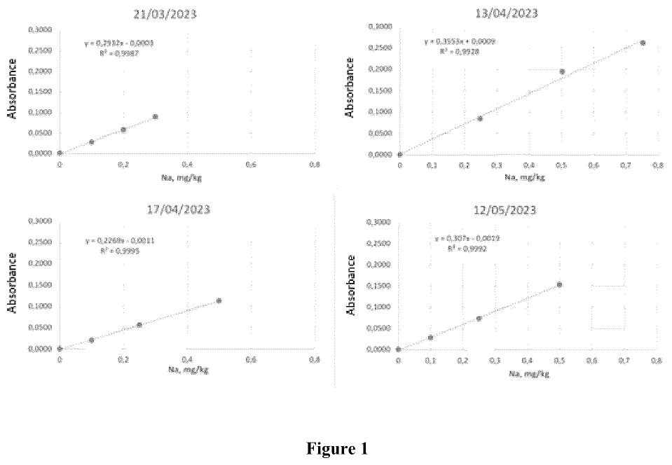

FIG. 1 shows the calibration curves for sodium diluted in a mixture of 3 g of ethanol-7 g of xylene, prepared on different days.

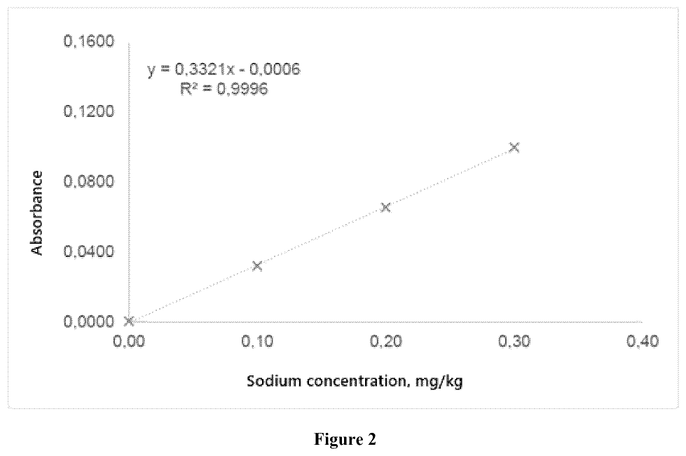

FIG. 2 shows the calibration curves for sodium diluted in a mixture of 3 g of ethanol-7 g of xylene, prepared on different days (average data).

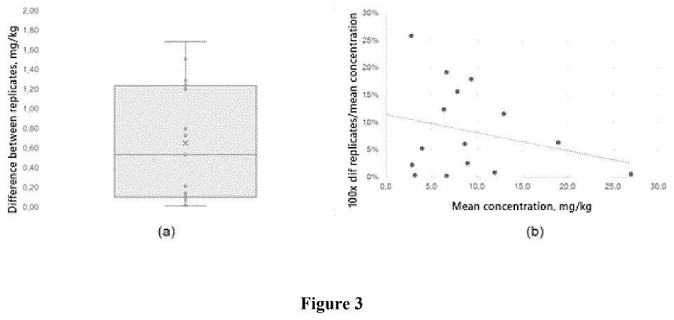

FIG. 3 shows in (a) the difference between the results of the replicates, and in (b) the % difference between replicates on the result found.

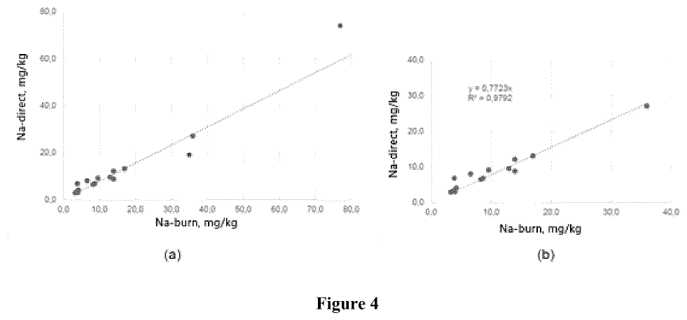

FIG. 4 shows the comparison of results between the burning method and the direct method, in (a) with results from samples outside the trend (2022-017437-79 and 2021-010428-76), and in (b) highlighting the fourteen results within the general trend.

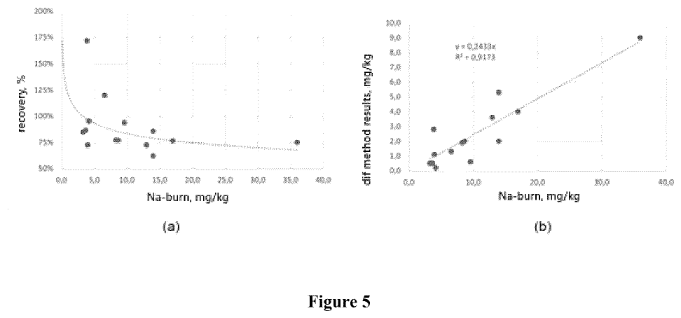

FIG. 5 shows in (a) the sodium recovery between the direct method and the burning method, and in (b) the absolute difference between the results of the direct method and the burning method.

### DETAILED DESCRIPTION OF THE INVENTION

The present invention relates to a direct method for the quantification of sodium in crude oil residues, such as atmospheric residues and vacuum residues, by flame atomic absorption spectrometry (FAAS).

The method uses a mixture of xylene and ethanol solvents to prepare the oil samples. The solvent mixture used ensures satisfactory recovery of inorganic sodium in the tested samples (around 80%). The calibration of the atomic absorption spectrometer equipment is performed with standards prepared with the solvent mixture (xylene and ethanol) in the tested proportion from aqueous standards of sodium salts in aqueous medium. The method comprises the following steps:

(a) preparation of the oil samples;

(b) preparation of the Na solution standards;

(c) preparation of the calibration standards.

### Preparation of the Oil Samples

Heat the sample in an oven for 30 minutes at a temperature between 80° C. and 120° C. (sufficient to make the sample flow). Homogenize the sample for 2 minutes with a previously cleaned and decontaminated glass rod, with circular movements and carefully to avoid projections or losses of the sample. Using a Pasteur pipette, weigh between 0.15 and 0.25 g of sample into a 15 mL Falcon tube containing 6.8 g of xylene and 3.0 g of ethanol, recording the exact mass to the nearest 0.1 mg. Carefully close the vials and shake vigorously manually for two minutes or with the aid of a Turrax equipment for thirty seconds.

### Preparation of Na Solution Standards

In a Falcon tube, add 0.10 mL of 1.000 mg/L Na aqueous standard to 10 g of distilled ethanol and shake vigorously for two minutes manually or with the aid of a Turrax for thirty seconds.

Preparation of Calibration Standards (in Ethanol and xylene)

Add the following masses to 15 mL tubes:

TABLE 1
Masses used in the preparation of calibration standards

| | | | Distilled |
|---|---|---|---|
| | Concentration | Standard mass | ethanol |
| Standards | (mg/kg) | 10 mg/kg (g) | mass (g) |
| Blank | 0.000 | 0.00 | 3.00 |
| Standard 1 | 0.100 | 0.10 | 2.90 |
| Standard 2 | 0.250 | 0.25 | 2.75 |
| Standard 3 | 0.500 | 0.50 | 2.50 |
| Zero adjustment | 0.000 | 0.00 | 3.00 |
| Verification standard | 0.050 | 0.05 | 2.95 |
| Signal adjustment | 0.500 | 0.50 | 2.50 |

Bring each solution to 10 g with xylene and shake vigorously for two minutes manually or with the aid of a turrax for thirty seconds. It is recommended to prepare at least three blanks as these are used to adjust the instrument zero and as a blank between sample readings. Adjust the equipment according to the instructions of the manufacturer.

Install an air/acetylene burner in a nebulization chamber suitable for working with organic media. The liquid level in the nebulizer trap should be topped up with organic solvent (xylene or kerosene), if necessary. Install a hollow cathode sodium lamp, aligning it with the optical system.

Adjust the wavelength of 589.0 nm and slit of 0.5 nm and the air-acetylene flame in the equipment software. Light the flame and, using the blank, adjust

the nebulization rate, burner height and flame stoichiometry. Check the signal using the 0.50 mg/kg standard.

Start the analysis by aspirating the calibration curve (blank, standard 1, standard 2 and standard 3) and then the samples, alternating at regular intervals between the samples, blank, verification standard and curve standard (reading them as a sample).

The conditions used in the spectrometer are summarized in Table 2. For aspiration of solutions in organic media, nebulizer adjustment is critical and must be performed immediately before each analysis.

TABLE 2
Analytical conditions used for sodium
quantification in organic media by FAAS

| Element | Na | |
|---|---|---|
| Wavelength | 589.0 | nm |
| Slot | 0.5 | nm |
| Background correction (D₂) | Turned off | |
| Flame | Air/acetylene | |
| Stoichiometry | Oxidant | |
| Burner | 10 | mm |

The solvents used were: Xylene P.A. and distilled Ethanol (from Ethanol P.A.). Commercially available ethanol P.A. contains sodium levels above 1 mg/kg, but simple distillation with a balloon and column eliminates this contamination. The sodium standards were prepared from an aqueous solution containing 1.000 mg/kg. The initial dilutions up to a concentration of 10 mg/kg are prepared in distilled Ethanol. For calibration, dilutions are made in Xylene P.A. in the same proportions as those used in the samples for the concentration levels prepared for the curve, in addition to a blank of the solvents used (0.0 mg/kg). The samples are diluted with ethanol and then immediately with xylene in the proportions defined previously (3 g of ethanol to 7 g of the sum of sample and xylene). For waste samples, it is necessary to heat the samples so that they flow.

### Calibration Curves

Calibration curves are used to adjust the equipment response, absorbance signal, versus concentration for sodium. The mathematical relationship between the measured absorbance signal versus concentration is obtained by the least squares method. In this way, a linear mathematical relationship is obtained in the form: y=ax+b, where “y” is the absorbance signal given by the equipment, “x” is the concentration of the solution read (standard or sample), “a” is the angular coefficient and “b” is the linear coefficient. There is also the calculation of the coefficient of determination (R²) which is a measure of the adjustment of the model ranging from 0 (model does not apply to the data obtained) to 1 (model applies perfectly to the data obtained).

A fundamental assumption of the least squares method is that each point on the graph (including the point representing the blank) has a normal distribution (in the y direction) with a standard deviation estimated by sy/x and can therefore be used in place of the standard deviation of the blank reading (sb) in estimating the limit of quantification (LQ) and these two estimation methods should not differ significantly. In practice, multiple determinations of the blank are time-consuming and the use of sy/x, already obtained directly from the calibration curve prepared for the test, becomes advantageous (MILLER, 2010). The limits of quantification (LQ) were calculated using Equation 1:

$$xLQ = xb + 10 sy / x \qquad ( Equation 1 )$$

where: xLQ—quantification limit, xb—mean signal of the blanks and sb—sy/x parameter obtained from the analytical curve, equivalent to the standard deviation of the curve.

The final concentrations of the standard solutions were calculated for the range between 0.0 mg/kg (blank of the solvents used) and 0.75 mg/kg, prepared on different days. After routine adjustments of the equipment (alignment of the optical part, flame stoichiometry, aspiration rate), the absorbance values were read and the calibration curve was assembled with the absorbance versus concentration readings (FIG. 1).

Na in its inorganic form (present in the standards) is solubilized and maintained in ethanol and xylene in the proportion of 3 g of ethanol to 7 g of xylene. The solubility of sodium chloride (NaCl) in ethanol is 0.65 g/kg at room temperature, and therefore the solubility of Na in ethanol is approximately 260 mg/kg. Even considering the approximately three-fold dilution in xylene, the solubility of sodium in the ethanol/xylene solution would be more than 100 times greater than the highest point on the calibration curve (solubility 78 mg/kg and highest point on the curve 0.75 mg/kg). However, the mixture with xylene possibly has effects on this Na solubility. And in the case of real samples, on the other salts present or even the other organic components present.

The first curve was prepared in the reading range 0 to 0.3 mg/kg. Due to the 50-fold dilution, the relative concentration range in the samples would be 0 to 15 mg/kg. Since higher concentrations were observed in the tested samples, the range was first extended to readings of 0 to 0.75 mg/kg (0 to 37.5 mg/kg in the samples) and then adjusted to 0 to 0.50 mg/kg (0 to 25 mg/kg in the samples). Wider ranges encompass more sample concentrations without the need for dilution, but narrower ranges can provide better linearity and precision. Therefore, the 0.00-0.10-0.25 and 0.50 mg/kg curve was adopted for analysis of the real samples.

The LQ estimate was made with curves of 0-0.10-0.20-0.30 mg/kg, prepared on six different days. The average results are indicated below (FIG. 2). The calculated instrumental LQ is 0.03 mg/kg and the method LQ (considering a dilution of 0.2 g of sample to 10 g of final mass, i.e. 50 times) is 1.5 mg/kg (Table 3).

TABLE 3
Linear regression data for calibration curves
prepared on different days (mean data)

| Standard concentration (mg/kg) | Absorbance |
|---|---|
| 0.00 | 0.0002 |
| 0.10 | 0.0318 |
| 0.20 | 0.0653 |
| 0.30 | 0.0998 |
| angular coefficient | 0.3321 |
| linear coefficient | −0.0006 |
| r² | 0.9996 |
| sy/x. absorbance | 0.0010 |
| sy/x. mg/kg | 0.0031 |
| instrumental LQ. mg/kg | 0.0311 |
| Method LQ. mg/kg | 1.554 |

### Examples of the Invention

Twenty-five samples with previously analyzed Na content and with available mass for testing were selected: twenty samples of atmospheric distillation residues, four samples of vacuum residues and one petroleum sample.

The samples were prepared in duplicate. In each replicate, a mass of 0.2 g of sample was solubilized with 3.0 g of distilled ethanol and 6.8 g of xylene (final dilution of 50 times by mass). The residue samples need to be previously heated in an oven so that they can flow (usually at temperatures above 60° C.). Additional care must be taken when handling heated liquids.

Nine of the results were below the quantification limit of the method (<1.5 mg/kg), being one petroleum sample, one RV sample and eight RAT samples (Table 4).

TABLE 4
Sodium results (mg/kg) in diluted real samples

| | | | | | | Difference |
|---|---|---|---|---|---|---|
| | | | | | | between |
| | | Sample | Replicate | Replicate | Aver- | replicates |
| Request | SCAD | type | 1 | 2 | age | 1 e 2 |
| 157522 | 2022- | RAT | (<0.5) | (<0.5) | <1.5 | — |
| | 031807-71 | | | | | |
| 158451 | 2022- | RAT | (<0.5) | (<0.5) | <1.5 | — |
| | 015456-24 | | | | | |
| 158451 | 2022- | Petróleo | (<0.5) | (<0.5) | <1.5 | — |
| | 017520-93 | | | | | |
| 158451 | 2022- | RAT | (<0.5) | (<0.5) | <1.5 | — |
| | 009261-36 | | | | | |
| 157522 | 2022- | RAT | (<0.5) | (<0.5) | <1.5 | — |
| | 031806-90 | | | | | |
| 157522 | 2022- | RV | (<0.5) | (<0.5) | <1.5 | — |
| | 033236-37 | | | | | |
| 158451 | 2022- | RAT | (<0.5) | (<0.5) | <1.5 | — |
| | 011974-05 | | | | | |
| 158451 | 2022- | RAT | (0.5) | (0.5) | <1.5 | — |
| | 011948-13 | | | | | |
| 158451 | 2022- | RAT | (1.3) | (1.3) | <1.5 | — |
| | 012106-06 | | | | | |
| 157522 | 2022- | RAT | 3.2 | 2.4 | 2.8 | 0.7 |
| | 031809-33 | | | | | |
| 158451 | 2022- | RAT | 3.0 | 2.9 | 2.9 | 0.1 |
| | 010723-86 | | | | | |
| 158451 | 2022- | RAT | 3.2 | 3.2 | 3.2 | 0.0 |
| | 018212-49 | | | | | |
| 158451 | 2022- | RAT | 3.9 | 4.1 | 4.0 | 0.2 |
| | 011989-91 | | | | | |
| 158451 | 2022- | RAT | 6.0 | 6.8 | 6.4 | 0.8 |
| | 014940-27 | | | | | |
| 158451 | 2022- | RAT | 6.7 | 6.7 | 6.7 | 0.0 |
| | 011983-04 | | | | | |
| 158451 | 2022- | RV | 7.3 | 6.0 | 6.7 | 1.3 |
| | 027977-24 | | | | | |
| 158451 | 2022- | RAT | 7.2 | 8.5 | 7.9 | 1.2 |
| | 014976-38 | | | | | |
| 157522 | 2022- | RAT | 8.4 | 8.9 | 8.7 | 0.5 |
| | 031808-52 | | | | | |
| 158451 | 2022- | RV | 9.1 | 8.9 | 9.0 | 0.2 |
| | 027979-96 | | | | | |
| 158451 | 2022- | RAT | 10.2 | 8.6 | 9.4 | 1.7 |
| | 008453-06 | | | | | |
| 158451 | 2022- | RAT | 11.7 | 11.8 | 12.0 | 0.1 |
| | 009253-26 | | | | | |
| 158451 | 2021- | RV | 14.0 | 12.4 | 13.0 | 1.5 |
| | 021273-01 | | | | | |
| 158451 | 2022- | RAT | 19.6 | 18.4 | 19.0 | 1.2 |
| | 017437-79 | | | | | |
| 158451 | 2022- | RAT | 26.6 | 26.7 | 27.0 | 0.1 |
| | 018190-07 | | | | | |
| 158451 | 2021- | RAT | 74.0 | 79.0 | 77.0 | 5.0 |
| | 010428-76 | | | | | |

The difference between the replicates obtained, excluding sample 2021-010428-76, was on average 0.6 mg/kg, being approximately between 0.2 and 1.2 mg/kg (FIG. 3a). Taking into account the relatively small mass (200 mg per replicate), this dispersion is at an acceptable level, less than 15% in most cases (FIG. 3b).

Comparison with Results Obtained by the Burning Method

The nine sodium results below the quantification limit of the direct method (<1.5 mg/kg) agreed qualitatively with those obtained by the burning method, with no “false negatives” (i.e., results that when analyzed by the burning method showed results above 1.5 mg/kg) (Table 5).

TABLE 5
Sodium results (mg/kg) in real samples by
the direct method and by the burning method

| | Sample | | Na, mg/kg | | Na, mg/kg |
|---|---|---|---|---|---|
| SCAD | type | Request | direct | Request | burn |
| 2022-009261-36 | RAT | 158451 | <1.5 | 151756 | <0.5 |
| 2022-011974-05 | RAT | 158451 | <1.5 | 152147 | <0.5 |
| 2022-015456-24 | RAT | 158451 | <1.5 | 152853 | <0.5 |
| 2022-033236-37 | RV | 157522 | <1.5 | 157522 | 0.6 |
| 2022-012106-06 | RAT | 158451 | <1.5 | 152209 | 0.7 |
| 2022-011948-13 | RAT | 158451 | <1.5 | 152142 | 0.7 |
| 2022-031807-71 | RAT | 157522 | <1.5 | 157522 | 0.8 |
| 2022-017520-93 | Petroleo | 158451 | <1.5 | 153242 | 1.0 |
| 2022-031806-90 | RAT | 157522 | <1.5 | 157522 | 1.1 |

From the comparison of the sixteen results above the LQ of the direct method with the burning method (Table 5), two samples appear to deviate from the general trend (FIGS. 4a): 2022-017437-79 and 2021-010428-76.

The results of the other fourteen samples follow the line with a calculated slope of 0.7723 (FIG. 4b). This means that the recovery of sodium by the direct method is, in general, between 75 and 80% (77.23%) of the result by the method involving burning the samples. In most cases, the burning method returned results superior to the direct method, with the exceptions being samples 2022-011983-04, 2022-014976-38 and 2021-010428-76.

TABLE 6
Sodium results (mg/kg) in real samples by the direct method and
by the burning method. In bold, the samples whose direct result
was higher than the burning result. In bold and underlined, the
results that deviate from the general trend (FIG. 4a).

| | Sample | | Na, mg/kg | | Na, mg/kg |
|---|---|---|---|---|---|
| SCAD | type | Request | direct | Request | burn |
| 2022-031809-33 | RAT | 157522 | 2.8 | 157522 | 3.3 |
| 2022-010723-86 | RAT | 158451 | 2.9 | 152013 | 4.0 |
| 2022-018212-49 | RAT | 158451 | 3.2 | 153344 | 3.7 |
| 2022-011989-91 | RAT | 158451 | 4.0 | 152153 | 4.2 |
| 2022-014940-27 | RAT | 158451 | 6.4 | 152815 | 8.3 |
| 2022-011983-04 | RAT | 158451 | 6.7 | 152153 | 3.9 |
| 2022-027977-24 | RV | 158451 | 6.7 | 157505 | 8.7 |
| 2022-014976-38 | RAT | 158451 | 7.9 | 152815 | 6.6 |
| 2022-031808-52 | RAT | 157522 | 8.7 | 157522 | 14 |
| 2022-027979-96 | RV | 158451 | 9.0 | 157505 | 9.6 |
| 2022-008453-06 | RAT | 158451 | 9.4 | 151473 | 13 |
| 2022-009253-26 | RAT | 158451 | 12 | 151756 | 14 |
| 2021-021273-01 | RV | 158451 | 13 | 148077 | 17 |
| 2022-017437-79 | RAT | 158451 | 19 | 153221 | 35 |
| 2022-018190-07 | RAT | 158451 | 27 | 153336 | 36 |
| 2021-010428-76 | RAT | 158451 | 77 | 145832 | 74 |

Within the general trend, higher recoveries (but also a greater dispersion of results) are observed in concentrations below 10 mg/kg (FIG. 5a). The absolute difference between the results of the direct method and the burning method does not exceed 4 mg/kg in 12 of the samples analyzed (FIG. 5b).

Therefore, the direct method proposed in the present invention proved to be an alternative for rapid response of the Na content in RAT/RV, not requiring any instrumentation or material other than that usually available in laboratories and serving for a diagnosis on the same day of sampling of the sodium content contained in the streams of interest.

The results of the new tests performed confirm the viability of using this direct method for rapid quantification of sodium, previously demonstrated: it is a faster method than the one currently available for the quantification of Na in RAT and RV samples, which makes it possible to monitor the concentrations within the same day.

The need to use a ratio of 3 g of ethanol to 7 g of xylene and sample was confirmed. With a greater number of samples for the test to be performed, the laboratory team was also able to improve their techniques and resources to perform this test.

Homogenizing these waste samples is laborious, but essential to obtain reliable results, and the efficiency of this step must be accompanied by the preparation f replicates of each sample (at least duplicate). One of the positive effects found with these new experiments was the absence of “false negatives” (result of the direct method indicating a value lower than the LQ of <1.5 mg/kg in samples with concentration >1.5 mg/kg measured by the burning method).

The recovery found for the set of samples tested was approximately 75% of sodium. In addition to being closer to the value taken as a reference, this greater recovery distances the measured value from the LQ, which may also explain the non-occurrence of “false negatives” in this data set. This recovery associated with the shorter time elapsed between sampling and the result indicates the potential of the application for monitoring processes with the possibility of greater analytical frequency (more samples per day) and therefore the possibility of action and correction when there are deviations in the process.

1. A method for the quantification of sodium in petroleum residues comprising the following steps:
(a) preparation of petroleum samples;
(b) preparation of Na solution standards; and
(c) preparation of calibration standards;
wherein quantification is performed by flame atomic absorption spectrometry (FAAS).

2. The method according to claim 1, wherein in step (a), the sample is heated in an oven for 30 minutes at a temperature between 80° C. and 120° C., followed by homogenization for 2 minutes with a previously cleaned and decontaminated glass rod, with circular movements, subsequently weighed with the aid of a Pasteur pipette into a 15 mL Falcon tube previously containing 6.8 g of xylene and 3.0 g of ethanol between 0.15 and 0.25 g of sample, noting the exact mass to the nearest 0.1 mg, and finally, vigorously shaken for between 30 seconds and 2 minutes, preferably with the aid of a turrax for thirty seconds.

3. The method according to claim 1, characterized in that wherein in step (b), 0.10 mL of Na 1.000 mg/L aqueous standard are added in a Falcon tube to 10 g of distilled ethanol, shaken vigorously for between 30 seconds and 2 minutes, preferably with the aid of a turrax for thirty seconds.

4. The method according to claim 1, wherein in step (c), the calibration standards are prepared in ethanol and xylene to a concentration of 10 mg/kg, wherein the samples are diluted with ethanol and then with xylene, preferably 3 g of ethanol for 7 g of the sum of sample and xylene.

5. The method according to claim 1, wherein the analytical conditions used in an organic medium for quantifying sodium by flame atomic absorption spectrometry (FAAS) are: 589.0 nm wavelength, 0.5 nm slit, 10 mm burner, air/acetylene flame, and oxidizing stoichiometry.

6. Use of the method, as defined in claim 1, for application in the analysis of sodium in RAT, RV and petroleum to monitor the efficiency of desalting in refineries and the effects of residual salt in the refining processes.
